(19) United States
(12) Patent Application Publication
(10) Pub. No.: US 2010/0056924 A1
(43) Pub. Date: Mar. 4, 2010

(54) CONTROL AND DISPLAY OF ULTRASONIC MICROBUBBLE CAVITATION

(75) Inventor: Jeffry E. Powers, Bainbridge Island, WA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., Eindhoven (NL)

(21) Appl. No.: 12/515,222

(22) PCT Filed: Nov. 13, 2007
(86) PCT No.: PCT/IB07/54613

§ 371 Date: May 15, 2009

Related U.S. Application Data
(60) Provisional application No. 60/866,521, filed on Nov. 20, 2006.

(51) Int. Cl. A61B 8/14

(52) U.S. Cl. 600/458

(57) ABSTRACT

An ultrasonic diagnostic imaging system is used to insonify a subject infused with a microbubble contrast agent. At low energy levels stable cavitation occurs as the bubbles oscillate radially without breaking up. At higher energy levels the bubbles dissolve or break up, termed inertial cavitation. Echo signals from microbubbles are bandpass filtered to produce signal components in a subharmonic band, indicative of stable cavitation, and signal component in a higher harmonic band indicative of inertial cavitation. Detection of the mode of cavitation is used to automatically or manually control the mode of cavitation by controlling the transmitted acoustic energy of the system.

This invention relates to medical diagnostic ultrasound systems and, in particular, to ultrasound systems which can be optimized to dissolve blood clots and similar occlusions.

Ultrasound is finding ever-expanding uses in therapeutic applications. Among those under current investigation are the use of ultrasound to assist in localized drug delivery and for lysis of materials in the body which are to be broken up and removed such as blood clots in blood vessels. Experimentation is directed to understanding the efficacy of ultrasound alone in these applications and also with the assistance of microbubble pharmaceutical agents. For instance, a drug compound can be encapsulated in microbubble shells and injected into the bloodstream, which eventually find their way to a target organ or vessel. Upon arrival, ultrasound can be used to break the microbubble shells, releasing the drug compound at an intended delivery site in the body. Experiments have also been conducted with higher ultrasound energy which is sufficient to disrupt cellular membranes at the delivery site, hastening the introduction of the drug into the cells.

In lysis applications it has been shown that ultrasound combined with microbubbles, with or without a thrombolytic drug, can speed dissolution of blood clots in stroke, heart attacks, and other occlusive vascular diseases. Recent results indicate that there is an optimal acoustic pressure range which maximizes the therapeutic effect while minimizing unintended bioeffects. Bubbles that are subjected to an acoustic field undergo radial expansion and contraction in response to the acoustic pressure waves. At relatively low levels of acoustic energy, this vibration is stable and can continue for considerable time, which has been demonstrated to maximize clot dissolution. This phenomenon is referred to as stable cavitation. At higher acoustic pressures, the bubbles become unstable and break into smaller bubbles and dissolve into the surrounding fluid, referred to as inertial cavitation. At even higher acoustic pressures the expansion of the bubbles can rupture very small capillaries containing the rupturing bubbles. The physiological mechanisms causing the enhanced therapeutic effect at the lower energy level are not fully understood. One commentator has speculated that the oscillation of the bubbles promotes local microstreaming of a surrounding pharmaceutical compound, increasing the penetration of the compound into an adjacent clot matrix. See “Mechanism Responsible for Ultrasound-accelerated Fibrinolysis in the Presence and Absence of Optison™,” by A. F. Prokop et al., presented at the 2006 IEEE International Ultrasonics Symposium (October 2006) and “Correlation of Cavitation With Ultrasound Enhancement of Thrombolysis,” by S. Datta, et al., Ult. in Med. & Biol., vol. 32, no. 8, p 1257-1267 (2006).

The two different forms of cavitation produce ultrasonic backscatter of different characteristics. Stable cavitation produces a strong subharmonic response, while unstable, or inertial cavitation produces broadband noise. These characteristics are used in passive cavitation detection in research situations to differentiate the two types of cavitation. Cavitation producing subharmonics is identified as stable, and cavitation causing broadband noise is identified as inertial. This is typically done with two separate transducers to transmit and receive in an in vitro situation. It would be desirable for an ultrasound system to controllably operate in the desired cavitation mode, either automatically or under manual user control.

In accordance with the principles of the present invention, a diagnostic ultrasound system and method are described which are operable either automatically or manually in a predetermined cavitation mode. Received echoes are analyzed by frequency and a significant subharmonic response is identified as stable cavitation. The analysis may compare the subharmonic response with a harmonic response to identify stable cavitation. In a manual mode the degree of stable or inertial cavitation is indicated to a user, enabling the user to adjust the transmitted energy for the desired cavitation. In an automatic mode the transmitted energy is adjusted automatically to acquire or maintain the desired cavitation.

In the drawings:

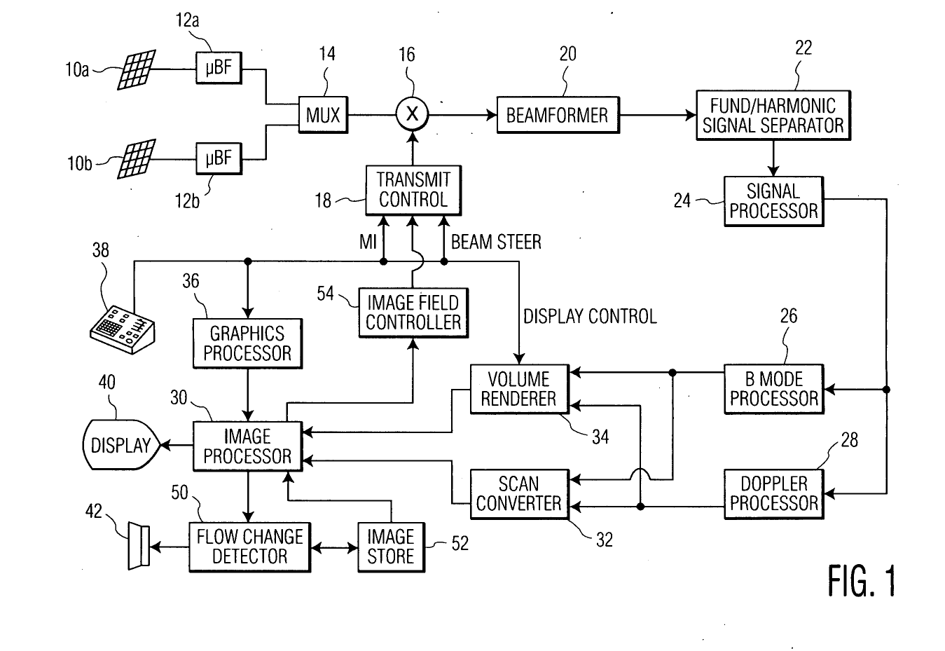

FIG. 1 illustrates in block diagram form an ultrasonic diagnostic imaging system constructed in accordance with the principles of the present invention.

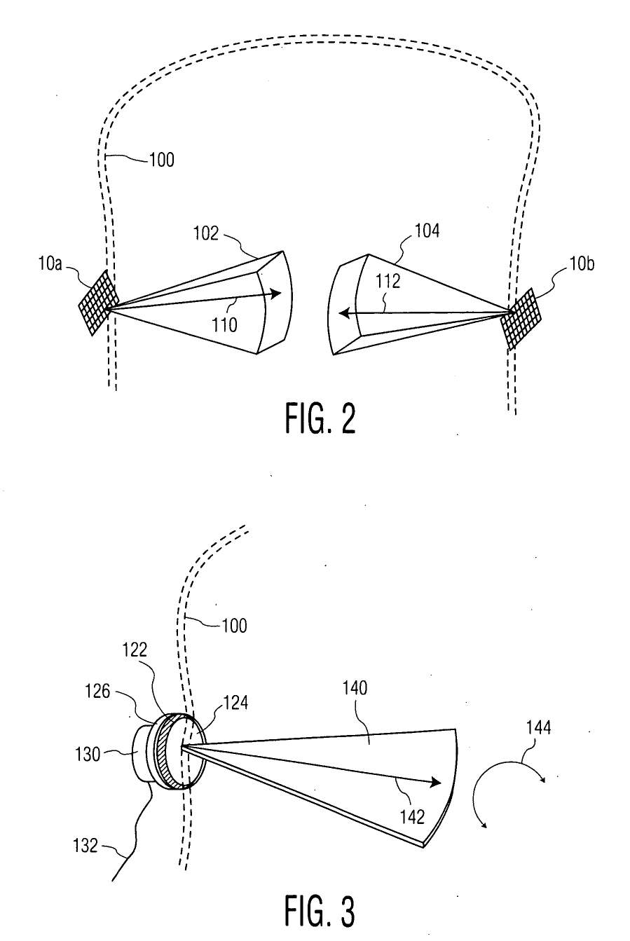

FIG. 2 illustrates three dimensional ultrasonic transcranial diagnosis in accordance with the present invention.

FIG. 3 illustrates two dimensional ultrasonic transcranial diagnosis in accordance with the present invention.

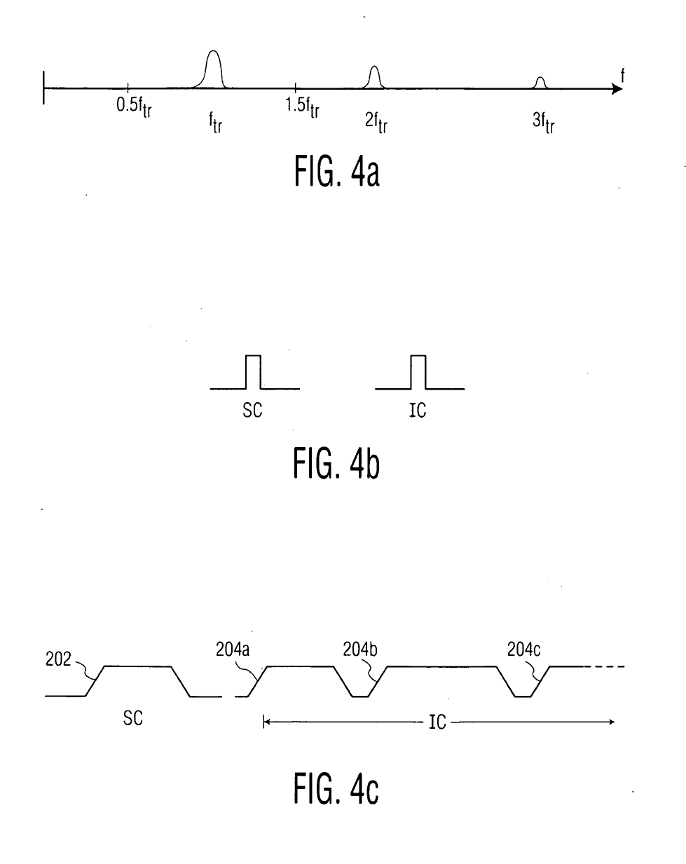

FIGS. 4a-4c illustrate received ultrasound signals and passbands which may be employed in an implementation of a system of the present invention.

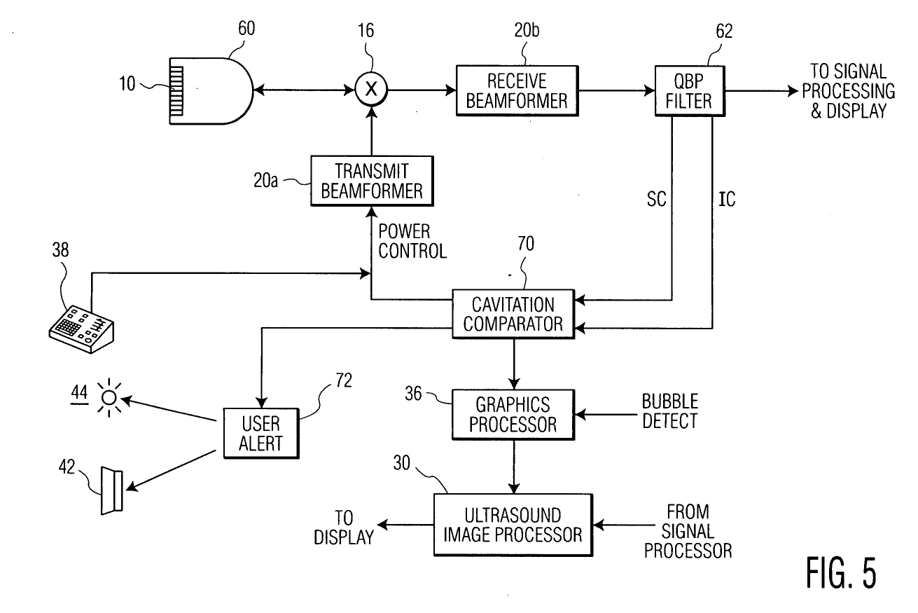

FIG. 5 illustrates in block diagram form a portion of an ultrasound system constructed in accordance with the principles of the present invention.

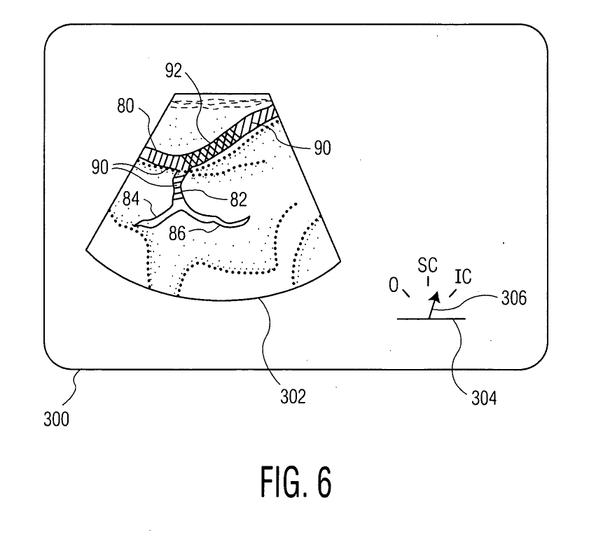

FIG. 6 is an example of a display produced by an ultrasound system or method in accordance with the principles of the present invention.

Referring first to FIG. 1, an ultrasound system constructed in accordance with the principles of the present invention is shown in block diagram form. The example of FIG. 1 is a system for diagnosing and treating cranial blood clots. The present invention may be employed to treat occlusions in other parts of the body such as myocardial, peripheral vascular, and renal occlusions. In FIG. 1 two transducer arrays 10a and 10b are provided for transmitting ultrasonic waves and receiving echo information. In this example the arrays shown are two dimensional arrays of transducer elements capable of scanning in three dimensions for 3D imaging and treatment, although an implementation of the present invention may also use one dimensional arrays of transducer element which produce 2D (planar) images. The transducer arrays are coupled to microbeamformers 12a and 12b which control transmission and reception of signals by the array elements. Microbeamformers are also capable of at least partial beamforming of the signals received by groups or “patches” of transducer elements as described in U.S. Pat. No. 5,997,479 (Savord et al.), U.S. Pat. No. 6,013,032 (Savord), and U.S. Pat. No. 6,623,432 (Powers et al.) Signals are routed to and from the microbeamformers by a multiplexer 14 by time-interleaving signals. The multiplexer is coupled to a transmit/receive (T/R) switch 16 which switches between transmission and reception and protects the main beamformer 20 from high energy transmit signals. The transmission of ultrasonic beams from the transducer arrays 10a and 10b under control of the microbeamformers 12a and 12b is directed by the transmit controller 18 coupled to the T/R switch, which received input from the user's operation of the user interface or control panel 38.

The partially beamformed signals produced by the microbeamformers 12a, 12b are coupled to a main beamformer 20 where partially beamformed signals from the individual patches of elements are combined into a fully beamformed signal. For example, the main beamformer 20 may have 128 channels, each of which receives a partially beamformed signal from a patch of 12 transducer elements. In this way the signals received by over 1500 transducer elements of a two dimensional array can contribute efficiently to a single beamformed signal.

The beamformed signals are coupled to a fundamental/harmonic signal separator 22. The separator 22 acts to separate linear and nonlinear signals so as to enable the identification of the strongly nonlinear echo signals returned from microbubbles. The separator 22 may operate in a variety of ways such as by bandpass filtering the received signals in fundamental frequency and harmonic frequency bands, or by a process known as pulse inversion harmonic separation. A suitable fundamental/harmonic signal separator is shown and described in international patent publication WO 2005/074805 (Bruce et al.) The separated signals are coupled to a signal processor 24 where they may undergo additional enhancement such as speckle removal, signal compounding, and noise elimination.

The processed signals are coupled to a B mode processor 26 and a Doppler processor 28. The B mode processor 26 employs amplitude detection for the imaging of structures in the body such as muscle, tissue, and blood cells. B mode images of structure of the body may be formed in either the harmonic mode or the fundamental mode or a combination of both as described in U.S. Pat. No. 6,283,919 (Roundhill et al.) and U.S. Pat. No. 6,458,083 (Jago et al.) Tissues in the body and microbubbles both return both types of signals and the stronger harmonic returns of microbubbles enable microbubbles to be clearly segmented in an image in most applications. The Doppler processor processes temporally distinct signals from tissue and blood flow for the detection of motion of substances in the image field including microbubbles. The structural and motion signals produced by these processors are coupled to a scan converter 32 and a volume renderer 34, which produce image data of tissue structure, flow, or a combined image of both characteristics. The scan converter will convert echo signals with polar coordinates into image signals of the desired image format such as a sector image in Cartesian coordinates. The volume renderer 34 will convert a 3D data set into a projected 3D image as viewed from a given reference point as described in U.S. Pat. No. 6,530,885 (Entrekin et al.) As described therein, when the reference point of the rendering is changed the 3D image can appear to rotate in what is known as a kinetic parallax display. This image manipulation is controlled by the user as indicated by the Display Control line between the user interface 38 and the volume renderer 34. Also described is the representation of a 3D volume by planar images of different image planes, a technique known as multiplanar reformatting. The volume renderer 34 can operate on image data in either rectilinear or polar coordinates as described in U.S. Pat. No. 6,723,050 (Dow et al.) The 2D or 3D images are coupled from the scan converter and volume renderer to an image processor 30 for further enhancement, buffering and temporary storage for display on an image display 40.

A graphics processor 36 is also coupled to the image processor 30 which generates graphic overlays for displaying with the ultrasound images. These graphic overlays can contain standard identifying information such as patient name, date and time of the image, imaging parameters, and the like, and can also produce a graphic overlay of a spatial indication of cavitation as described below. For these purposes the graphics processor receives input from the user interface 38. The user interface is also coupled to the transmit controller 18 to control the generation of ultrasound signals from the transducer arrays 10a and 10b and hence the images produced by and therapy applied by the transducer arrays. The transmit parameters controlled in response to user adjustment include the MI (Mechanical Index) which controls the peak intensity or power of the transmitted waves, which is related to cavitational effects of the ultrasound, steering of the transmitted beams for image positioning and/or positioning (steering) of a therapy beam as discussed below. As explained in greater detail below, the transmit power or MI is controlled to determine the type of microbubble cavitation produced.

The transducer arrays 10a and 10b transmit ultrasonic waves into the cranium of a patient from opposite sides of the head, although other locations may also or alternately be employed such as the front of the head or the sub-occipital acoustic window at the back of the skull. The sides of the head of most patients advantageously provide suitable acoustic windows for transcranial ultrasound at the temporal bones around and above the ears on either side of the head. In order to transmit and receive echoes through these acoustic windows the transducer arrays must be in good acoustic contact at these locations which may be done by holding the transducer arrays against the head with a headset. One acceptable way to do this is with a headset as described in my U.S. patent application 60/822,106 filed Aug. 11, 2006, the contents of which is incorporated herein by reference. With the transducer arrays in good acoustic contact with the temple regions of the skull, 3D acoustic fields 102 and 104 can be scanned as shown in FIG. 2. In the depiction of FIG. 2, each image field 102, 104 is seen to extend almost halfway across the cranium, which is a balance between the size of the image field and the acoustic penetration and attenuation which may be expected through the bone at the temporal acoustic window. For some patients, low attenuation effects may enable an image field to extend fully across the cranium, allowing the clinician to examine the vascular structure near the skull bone on the opposite side of the cranium. By alternately examining image fields of both transducer arrays, the vasculature across the full cranium may be effectively examined. It is possible to acquire extended image fields which cover the same central region of the cranium but image from opposite sides of the head. These images can be correlated and compounded together, forming a fused image that may reveal additional characteristics of the brain. If examination finds a blood vessel occluded by a blood clot, a therapeutic beam 110 or 112 is transmitted and steered at the clot to break it up either with the ultrasonic energy alone, but preferably in combination with microbubbles and a thrombolytic drug such as tissue plasminogen activator (tPA). The therapeutic beam 110,112 can also be transmitted from both sides of the head, enabling breakup of a clot from both sides of the clot. Rather than be limited to reflective ultrasound imaging, through-transmission imaging can be performed by transmitting ultrasound from one transducer array and receiving the remaining unabsorbed ultrasonic energy at the other transducer array, which may reveal yet other characteristics of the brain tissue.

FIG. 3 illustrates a two dimensional imaging example of the present invention. In this example the transducer array 122 is a one dimensional array which performs 2D imaging. The array is configured as a circular phased array transducer as described in U.S. Pat. No. 5,226,422. This transducer array, like the other arrays described herein, is covered with a lens 124 which electrically insulates the patient from the transducer array and in the case of a one dimensional array may also provide focusing in the elevation (out-of-plane) dimension. The transducer array 122 is backed with acoustic damping material 126 which attenuates acoustic waves emanating from the back of the array to prevent their reflection back into the transducer elements. Behind this transducer stack is a device 130 for rotating the image plane 140 of the array. The device 130 may be a simple knob or tab which may be grasped by the clinician to manually rotate the circular array transducer in its rotatable transducer mount (not shown). The device 130 may also be a motor which is energized through a conductor 132 to mechanically rotate the transducer as discussed in U.S. Pat. No. 5,181,514 (Solomon et al.) Rotating the one dimensional array transducer 122 as indicated by arrow 144 will cause its image plane 140 to pivot around its central axis, enabling the repositioning of the image plane for full examination of the vasculature in front of the transducer array. As discussed in the '514 patent, the planes acquired during at least a 180° rotation of the array will occupy a conical volume in front of the transducer array, which may be rendered into a 3D image of that volumetric region. Other planes outside this volumetric region may be imaged by repositioning, rocking or tilting the transducer array in its headset in relation to the skull 100. If a stenosis is found in the image of the plane being imaged, the therapeutic beam vector graphic 142 can be steered by the clinician to aim the therapeutic beam at the stenosis and therapeutic pulses applied to disrupt the microbubbles at the site of the stenosis.

In accordance with the principles of the present invention the therapeutic beam is modulated to produce an optimal therapeutic effect, one which breaks up the blood clot quickly and effectively through agitation of neighboring microbubbles. FIG. 4a illustrates a range of frequencies for a transmit pulse or wave and its echo components. In a typical implementation the therapeutic transmit pulse fₜᵣ is a long pulse (long sample volume) directed at a blood clot. As explained in my previously filed application 60/822,106, longer transmit pulses have been found to have a more beneficial therapeutic effect. One reason for this may be that the oscillating bubbles are sustained longer at the locus of the therapy through rectified diffusion. In addition, longer pulses can be tailored to contain a narrower range of frequency components than short pulses, which exhibit a broader range of frequencies. This permits the use of narrower filters with more precise control in an implementation of the present invention. While monotonic long pulses can be employed, this will generally result in reduced image resolution. Image resolution can be improved by the use of long pulses which are coded, such as long pulses with frequency modulation. An example of such a pulse is a chirp waveform.

When a transmit pulse fₜᵣ is reflected by a microbubble the reflected echo will have significant harmonic content due to the nonlinear behavior of the microbubble in the acoustic field. The second harmonic component 2fₜᵣ and the third harmonic, 3fₜᵣ, are also illustrated in FIG. 4a. The transmitted wave will also be distorted by its passage through tissue which will also generate harmonic echo returns, but generally at lower levels than the harmonic echo returns from microbubbles. The tissue harmonic components are most prevalent at the integer harmonic frequencies 2fₜᵣ and 3fₜᵣ, with successively higher harmonics being of decreasing amplitude.

At different transmit power (MI) levels microbubbles will exhibit different behavior. As previously mentioned, bubbles in the body will exhibit radial expansion and contraction in response to the acoustic pressure waves. At relatively low levels of acoustic energy, this oscillation is stable and can continue for a considerable amount of time. This continual agitation by the oscillating bubbles may be a phenomenon that contributes to the effectiveness of the bubbles in breaking up a clot, and is referred to as stable cavitation. Stable cavitation is maintained by maintaining the energy of the acoustic pressure waves at an intensity which produces this effect. At higher acoustic pressures, the bubbles become unstable and break into smaller bubbles and dissolve into the surrounding blood, and at even higher acoustic pressures even within diagnostic power limits the bubbles can rupture violently and disappear. This removal of the bubbles may be a factor contributing to the relative ineffectiveness of these inertial cavitation pressure levels in promoting clot dissolution. Thus it is desirable to maintain the transmit acoustic energy at a level which sustains stable cavitation without the onset of significant inertial cavitation.

As previously mentioned bubbles which are in stable cavitation will return echo signals with significant subharmonic content below the fundamental transmit frequency fₜᵣ. For instance, echoes from stable cavitation can be expected to return echo signals with frequencies at a frequency of 0.5fₜᵣ as indicated in FIG. 4a. The frequency content of echoes can be analyzed to determine whether there is significant frequency content about this subharmonic frequency. A bandpass filter with a center frequency of 0.5fₜᵣ will perform this function as indicated in FIG. 4b, in which a passband centered at this frequency will pass signal energy about this frequency. The energy content of this SC passband can be examined for significant subharmonic energy content and used as an indication of stable cavitation. The transmit power of the acoustic energy is then adjusted or maintained to maximize the energy content of the SC passband, thereby establishing and maintaining stable cavitation. The analysis of the SC passband can be performed with respect to a predetermined threshold or by adjusting the transmit energy until a maximum response in the SC passband is produced. The SC passband energy can also be compared with the energy of a frequency spectrum characteristic of inertial cavitation. For instance, since a signature of inertial cavitation is broadband frequency content above the fundamental frequency fₜᵣ, a passband can be established at these higher frequencies such as the IC passband illustrated in FIG. 4b at 1.5fₜᵣ. This fractional (non-integer) harmonic frequency is used to reduce contributions from tissue harmonic echo returns, which exhibit significant frequency content at integer harmonic frequencies such as the second harmonic 2fₜᵣ. One technique of using both frequencies is to adjust the transmit power until a maximum ratio of the SC band energy to the IC energy is obtained. Another approach is to increase transmit power to a level just below the occurrence of significant or detectable energy in the IC band. Thus, stable cavitation is maintained without the onset of undesired inertial cavitation. This is of considerable benefit in transcranial applications because the skull is highly attenuative and varies from person to person. It is very difficult to predict in advance the transmit power level needed to maintain stable cavitation in the head of a particular patient. The present invention solves this problem by detecting and identifying the type of cavitation then allowing the user to automatically or manually control the cavitation mode.

Yet another approach is to use a wider passband in order to be more sensitive to energy at subharmonic and higher harmonic frequencies as shown in FIG. 4c. In this example the SC passband 202 rolls off below 0.5 fₜᵣ at the low end and just before the fundamental frequency fₜᵣ. The cutoff can be fairly sharp as frequency bands can be more tightly controlled with longer transmit pulses with their characteristically narrower frequency content. For inertial cavitation detection a broader passband is used to take advantage of the wide frequency content returned from inertial cavitation events. In this example the first sub-band 204a of the IC band has a lower cutoff above the fundamental frequency fₜᵣ and below 1.5 fₜᵣ. The passband is notched out at the second harmonic 2fₜᵣ and continues with a second sub-band 204b above the second harmonic frequency and below the third harmonic frequency 3fₜᵣ. Additional sub-bands such as 204c can also be used. This IC passband captures the broadband energy of inertial cavitation events with reduced response to tissue harmonic energy at the integer harmonic frequencies.

A further approach is to use a narrower passband in the subharmonic range and a broader passband in the harmonic range. For example a narrow passband such as the SC passband of FIG. 4b can be used for stable cavitation detection, and a broader passband such as passband 204a of FIG. 4c can be used for inertial cavitation detection.

FIG. 5 is a detailed block diagram of one implementation of the concepts of the present invention in the ultrasound system of FIG. 1. An ultrasound probe 60 has a transducer array 10 coupled by T/R switch 16 to a transmit beamformer 20a which controls the transmission of therapeutic beams by the transducer array 10 and to a receive beamformer 20b which beamforms echo signals received from the transducer array elements 10. The beamformed echo signals are processed by a quadrature bandpass (QBP) filter 62. QBP filters are commonly used in ultrasound systems to filter received echo signals, produce I and Q quadrature signal components for Doppler and coherent image processing and provide sampling decimation. QBP filters are generally described in U.S. Pat. No. 6,050,942 (Rust et al.), for example. In this implementation the QBP filter also filters the echo signals into two passbands, an SC band and an IC band such as those shown in FIG. 4. The signal content of the SC and IC bands is then analyzed by a cavitation comparator 70 which may analyze the SC signal content alone for its energy content or in comparison with a threshold level, or may analyze the SC signal content in comparison with the IC signal content as described previously. The result of the analysis will indicate the presence of stable cavitation, inertial cavitation, or both or neither.

The result of the cavitation analysis is used in an automated implementation to control the transmit energy level of the transducer array 10. This is done in this example by coupling a cavitation control signal from the cavitation comparator 70 to the power control input of the transmit beamformer 20a. When it is desired to produce stable cavitation of microbubbles the cavitation control signal will vary the transmit power until a maximum response is detected in the SC passband, at which point that transmit power level is maintained to maintain stable cavitation. Alternatively or additionally, the response of the SC passband can be compared with that of the IC passband and the transmit power level controlled to obtain the desired SC to IC band ratio.

In a manual implementation the user will control the transmit power from the transmit power control of the user interface 38. As the user increases power from a low power setting with microbubbles present, stable cavitation will begin to occur, producing subharmonic energy in the SC passband which is detected by the cavitation comparator, either alone or in combination with higher frequency energy of the IC passband. When stable cavitation is identified by the cavitation comparator 70 a control signal is coupled to a user alert 72 which issues an audible or visual alert to the user. The audible alert can comprise a tone of a given frequency or amplitude from a speaker 42 when stable cavitation is detected, and can change to or be mixed with a tone of a different frequency or sound when inertial cavitation is detected. The user will thus adjust the power level until the stable cavitation tone is continuously heard without interruption by the inertial cavitation tone. Alternatively or in addition, a visual indication 44 can be presented, such as a green light when stable cavitation is detected and changing to a red light when inertial cavitation is present. The user will adjust the power for a solid green light in that example.

Another approach is illustrated in the ultrasound system display screen 300 of FIG. 6, which shows the presence of cavitation spatially. The display 300 is displaying an ultrasound image 302 which includes tissue and vasculature. The vasculature includes a major blood vessel 80, off of which is branching a smaller vessel 82 which branches into smaller capillaries 84 and 86. The blood vessels will appear black in grayscale due to the low level echo returns from the blood, but can be shown in color in Doppler mode with the color indicating the motion of the blood. As the probe 60 scans the region of the body producing the image, the B mode signals from the vessels will change from black (low level) to bright (high level) when the blood flow begins to contain appreciable amounts of microbubbles. The arrival of microbubbles in the blood flow can be detected adaptively from this change or the system can be baselined manually by placing a sample volume over a vessel without microbubbles and measuring the signal content, then placing the sample volume over a vessel when microbubbles are present to measure the signal content from the microbubbles. Either the adaptive or manual technique can be used, in conjunction with the flow (Doppler) information if desired, to detect signals coming from microbubbles. When a microbubble has been detected at a spatial location in the image, the “bubble detect” signal triggers the graphics processor 36 in FIG. 5 to place a color in a color overlay of the image 302 which indicates the type of cavitation at each bubble location. The color is determined by a cavitation signal coupled to the graphics processor from the cavitation comparator 70. For instance, if the SC signal or SC/IC ratio indicates the presence of stable cavitation at a location where a bubble is detected, a green color is added to the overlay at that bubble location, as indicated by the single hatching 90 in FIG. 6. But when inertial cavitation is detected by the cavitation comparator, a different color is added to the overlay such as a red color at that bubble location. The red color is indicated by cross-hatching 92 in FIG. 6. When no cavitation is detected as will occur in signals returned from tissue or blood without microbubbles, no overlay color is added and the ultrasound image will appear conventional at those locations. The cavitation color overlay is combined with the ultrasound image by the ultrasound image processor 30.

In the example of FIG. 6 it is seen that stable cavitation (single hatching 90) is occurring in the lateral extremes of the major vessel 80 and into the smaller vessel 82, but that inertial cavitation (cross-hatching 92) is occurring in the center of the major vessel 80. A user trying to maintain stable cavitation will then turn down the transmit power until the red color 92 is replaced with the green color 90, indicating the presence of only stable cavitation in the vasculature. This would be an optimal operating condition to dissolve a blood clot located at the junction of the major vessel 80 and the smaller vessel 82, for instance.

FIG. 6 also shows a display indicator 304 which displays the type of cavitation detected. The pointer 306 of the display indicator can point at zero, SC, or IC or between these indications. The instantaneous setting of the pointer 306 is determined by a signal from the cavitation comparator to indicate the predominant type of cavitation detected. If no cavitation is detected in the image the pointer will point to zero. As stable cavitation begins to manifest itself the pointer will move to the SC indication, and if the power is turned too high and inertial cavitation begins, the pointer will move to the IC indication. The pointer can indicate an average or overall cavitation content of the vasculature by summing or integrating the cavitation signals over the points in the image where microbubbles have been detected. The user adjusts the transmit power level to keep the pointer 306 pointing continually at the SC indication.

Other variations using the concepts of the present invention will occur readily to those skilled in the art. For instance, a certain clinical application can call for inertial cavitation as the preferred mode and an implementation of the present invention can be used to control or maintain that mode. For instance, the user can be operating in a “flash” mode in which it is desired to quickly destroy the microbubbles in the image region and image this destruction, or to observe the build-up of microbubbles as a new flow of microbubbles return to the bubble-depleted image region. In this example the user will try to produce the audible or visual indication of inertial cavitation without stable cavitation during and immediately following the “flash” of bubble-breaking acoustic energy.

What is claimed is:

1. An ultrasonic diagnostic imaging system which controls microbubble cavitation comprising:
a transducer array which operates to transmit and receive echo signals from a region of a subject which contains microbubbles;
a transmitter coupled to the transducer array with a power control input which acts to control the acoustic energy level transmitted by the transducer array;
a cavitation processor coupled to analyze echo signals from microbubbles for subharmonic frequency content,
wherein the identification of subharmonic frequency content is used to control the cavitation mode of the microbubbles.

2. The ultrasonic diagnostic imaging system of claim 1, wherein the identification of subharmonic frequency content is used to manually control the cavitation mode of the microbubbles.

3. The ultrasonic diagnostic imaging system of claim 1, wherein the identification of subharmonic frequency content is used to automatically control the cavitation mode of the microbubbles.

4. The ultrasonic diagnostic imaging system of claim 3, wherein the frequency analyzer is coupled to the power control input of the transmitter.

5. The ultrasonic diagnostic imaging system of claim 4, wherein the acoustic energy level transmitted is maintained at a level designed to maintain cavitation in the stable mode.

6. The ultrasonic diagnostic imaging system of claim 1, further comprising a bandpass filter having an input coupled to the transducer array and an output coupled to the cavitation processor.

7. The ultrasonic diagnostic imaging system of claim 6, wherein the bandpass filter produces a first response at a subharmonic frequency and a second response at a harmonic frequency above the fundamental frequency,
wherein the first response is indicative of stable cavitation and the second response is indicative of inertial cavitation.

8. The ultrasonic diagnostic imaging system of claim 7, wherein the cavitation processor is responsive to the first and second filter responses for producing a control signal coupled to the power control input of the transmitter.

9. The ultrasonic diagnostic imaging system of claim 7, further comprising a user input coupled to the power control input of the transmitter,
wherein the cavitation processor is responsive to at least one of the first and second filter responses for actuating an audible or visual indication of the cavitation mode.

10. An ultrasonic diagnostic imaging system which displays microbubble cavitation comprising:
a transducer array which operates to transmit and receive echo signals from a region of a subject which contains microbubbles;
an image processor coupled to the transducer array which utilized the received echo signals to produce a spatial image of the region of the subject;
a cavitation detector coupled to receive echo signals which operates to detect at least one of stable or inertial microbubble cavitation,
wherein the cavitation detector is coupled to the image processor for indicating spatial locations in the image where cavitation is detected.

11. The ultrasonic diagnostic imaging system of claim 10, wherein the cavitation detector is further operable to indicate the locations in the image where stable and inertial cavitation are detected by distinguishing visual characteristics.

12. The ultrasonic diagnostic imaging system of claim 11, wherein the distinguishing visual characteristics comprise different colors.

13. The ultrasonic diagnostic imaging system of claim 12, wherein the cavitation detector and the image processor are further operable to indicate the locations in the image where stable and inertial cavitation are detected by a color overlay for a spatial ultrasonic image.

14. A method for controlling an ultrasound system to produce a desired mode of microbubble cavitation comprising:
detecting echo signals from regions in a diagnostic field where microbubbles are present;
analyzing the echo signals for the presence of at least one of stable or inertial cavitation; and
controlling the transmitted acoustic energy of the ultrasound system to produce the desired cavitation mode.

15. The method of claim 14, wherein analyzing further comprises analyzing subharmonic frequency signal content for the presence of stable cavitation.

16. The method of claim 15, wherein analyzing further comprises analyzing harmonic frequency signal content above the fundamental transmit frequency for the presence of inertial cavitation.

17. The method of claim 16, further comprising producing a user alert of the presence of at least one of stable or inertial cavitation,
wherein controlling further comprises manually controlling the transmitted acoustic energy.

18. The method of claim 16, further comprising producing a control signal in response to the detection of cavitation; and
coupling the control signal to a power control input of an acoustic energy transmitter.

19. A method for producing an ultrasound image which indicates the presence of microbubble cavitation on a spatial basis comprising:
receiving echo signals from an image region of a subject;
producing an ultrasound image of the image region in response to the echo signals;
detecting the presence of at least one of stable or inertial microbubble cavitation in the image region; and
producing an indication on the ultrasound image of a spatial location where microbubble cavitation is detected.

20. The method of claim 19, wherein producing further comprises coloring a spatial location of the ultrasound image where microbubble cavitation is detected.
